(19) United States
(12) Patent Application Publication
(10) Pub. No.: US 2020/0352779 A1
(43) Pub. Date: Nov. 12, 2020

(54) PROGRAMMABLE COLD THERAPY SYSTEM

(71) Applicant: Polar Products, Inc., Stow, OH (US)

(72) Inventor: WILLIAM S. GRAESSLE, Hudson, OH (US)

(21) Appl. No.: 16/408,608

(22) Filed: May 10, 2019

(51) Int. Cl. A61F 7/00; A61F 7/02; A61F 7/10

(52) U.S. Cl. CPC A61F 7/0085; A61F 7/02; A61F 7/10; A61F 2007/0093; A61H 2201/5007; A61H 2201/0214; A61F 2007/0056

(57) ABSTRACT

A programmable cold therapy system is employed for pain relief and healing following injury or surgery. The system has a tank that maintains a reservoir of ice water. A pump is received within the tank and draws cold water from the reservoir to a cooling pad positioned over a region of the patient's or user's body requiring cold therapy. A control panel selectively interconnects a power source with the pump to control a period of time that the pump may be operative, and further controls periods of time during which the pump is actually operative. Within a first period of time, the pump is enabled, while it is actually actuated during repetitive second periods of time to operate at a selected duty cycle.

### FIELD OF THE INVENTION

The invention herein resides in the art of therapeutic systems and devices arid, more particularly, to a system for delivering drug-free pain relief to individuals or patients following injury or surgery. More particularly, the invention relates to such devices and systems that provide such pain relief through the application of compression wraps to various parts of the human body. Specifically, the invention relates to a cold therapy system by which a fluid, such as water, circulates between a body region to be treated by cold therapy and a reservoir where the fluid may be cooled. More specifically, the invention relates to a cold therapy system that is programmable as to the duration of cold therapy treatment as to time, temperature, sequence and duration,

### BACKGROUND OF THE INVENTION

The management of pain following injury or surgery has been a matter of attention in the medical field for decades, Known methodologies for the management of pain require the use of drugs, the most effective of which are often addictive/habit-forming. While the use of such drugs may be effective during recovery and treatment, if their use is not carefully monitored, controlled and restricted, the serious consequence of addiction often follows.

Drug-free pain relief is most desirable for treating injuries and effecting pain management post-surgery.

Cold therapy is an alternative methodology for effecting pain relief and hastening recovery without the use of opioids or other drugs. The use of cold packs, pads and the like on a targeted area is commonplace. Presently, there are known systems and units that provide a flow of cold water through a pad or bladder that may be placed over or secured to a region of the body requiring treatment. However, these known units are of a very rudimentary nature with little or no ability to control or regulate their use, Known units are simply of the on/off type, turning on when they are plugged into a wall outlet and turning off when unplugged. Medical professionals typically recommend an on/off sequence of treatment, precluding the application of cold therapy to the body for any prolonged continuous period. A substantive reduction of skin/flesh temperature can cause significant problems, particularly for those with circulatory conditions, diabetes, and the like. Unfortunately, during use of presently known systems that require the patient to turn the system on and off; unintended excessive or prolonged use is not uncommon. Quite frequently, the patient may fall asleep with the cold therapy system in operation, applying cold fluid through the pad or bladder for a prolonged period, significantly lowering the temperature of the affected area of the patient's skin and flesh. The cold therapy system then harms rather than helps the patient.

The prior known systems also do not provide any means for effecting on/off cycles (duty cycles) for regulating the flow of the cooling fluid through the pad or bladder, nor for regulating the rate of flow of that cooling fluid to control the effective temperature of the fluid at the pad or pack. Nor do known systems regulate the sequence and durations of the flow of the cooling fluid through the pad or bladder. Such features are absent in the art despite the fact that the medical profession does not adhere to a universally accepted protocol for using cold therapy after surgery. The duty cycles vary widely among physicians, with some simply directing to “use it as you like.”

### DISCLOSURE OF INVENTION

In light of the foregoing, it is a first aspect of the invention to provide a programmable cold therapy system.

Another aspect of the invention is to provide a programmable cold therapy system having the capability to regulate the time, temperature and duration of the application of cold therapy to art affected area of the human body.

Still another aspect of the invention is the provision of a programmable cold therapy system that departs from the traditional on/off system, but provides for tailoring and adaptability to meet the particular needs of the patient as prescribed by the patient's doctor.

Still a further aspect of the invention is the provision of a programmable cold therapy system in which a timer may be employed to effect the duration of a treatment session, the duty cycle within that session, and the temperature to which the treated area is exposed.

Yet another aspect of the invention is the provision of a programmable cold therapy system that is portable and adapted for use in any of a wide variety of environments.

Still another aspect of the invention is the provision of a programmable cold therapy system that relies only upon the user or operator to initiate the treatment and is self-controlling thereafter.

An additional aspect of the invention is the provision of a programmable cold therapy system that is effective and safe in use, eliminates the need for and risk attendant the use of opioid pain-killers, is cost effective, and can be readily made using state-of-the-art materials arid devices.

The foregoing and other aspects of the invention that will become apparent as the detailed description proceeds are achieved by a programmable cold therapy system for pain relief and promote healing after an injury or surgery, comprising: a tank for maintaining a reservoir of cooling fluid; a pump within said tank and in communication with said reservoir of cooling fluid; a cooling pad; a hose assembly interconnecting said cooling pad and pump; and a control panel interconnecting a power source and said pump, said control panel regulating operation of said pump.

Further aspects of the invention are achieved by a programmable cold therapy system for pain relief and to promote healing, comprising: a tank having a reservoir of ice water; a cooling pad having a bladder and selectively attachable to a portion of a user's body; a pump within said reservoir of ice water; a first hose interconnecting said pump with said bladder; a second hose interconnecting said bladder with said reservoir; and means for operating said pump at a selected duty cycle within a selected window of time.

### BRIEF DESCRIPTION OF THE DRAWINGS

For a complete understanding of the various aspects of the invention, reference should be made to the following detailed description and accompanying drawings wherein:

FIG. 1 is an illustrative presentation of the programmable cold therapy system of the invention shown in use with a patient demonstrating cold therapy applied to a shoulder; and

FIG. 2 is an illustrative schematic diagram of the structure accommodating the programmable features of the invention.

### DETAILED DESCRIPTION OF ILLUSTRATIVE EMBODIMENTS

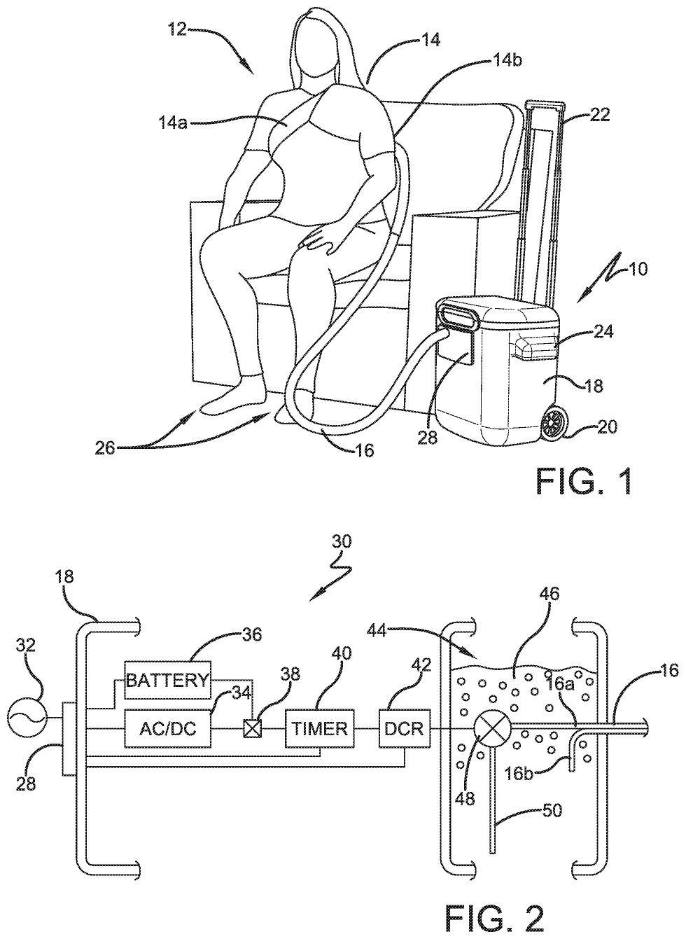

Referring now to the drawings and more particularly FIG. 1, it can be seen that a programmable cold therapy system made and used in accordance with the concepts of the invention is designated generally by the numeral 10. The system 10 is shown as treating a post-surgery patient 12 or other individual requiring cold therapy for an injury or the like. A shoulder cooling pad or bladder 14 is shown as positioned on the left shoulder of the patient 12 and secured thereto by appropriate straps 14a, 14b or the like. A paired tubing 16, maintains cooling and return tubes to be discussed later.

A thermally insulated tank 18 has a hinged lid to allow access to the interior thereof and is adapted to receive and maintain a volume of ice and water suitable for intended purposes. The tank 18 may be mobile as by wheels 20, a telescoping handle 22, and fixed handles 24.

As presented above, the system 10 shown in FIG. 1 is adapted to effect cold therapy on the shoulder of the patient 12 by the passage of cooling fluid, such as ice water, through the paired tubing 16 to the shoulder cooling pad or pack 14 and the return thereof through the paired tubing 16 to the tank 18. The invention also contemplates that the paired tubing 16 may be interconnected with any of a host of other cooling pads beyond that employed for the shoulder. For instance, ankle cooling pads 16 may be positioned about the ankle of a user who has had surgery, sprains, tendinitis, or other similar maladies in the ankle area. Devices of the nature contemplated by the instant invention include not only those for the shoulder and ankle, but also for hands and wrists, legs, thighs, knees, calves, head, back and the like.

The cooling pads have bladders, often serpentine in nature, through which the cooling fluid or ice water passes in close juxtaposition to the body part to be treated. These pads or packs are held in place by appropriate securing means such as Velcro® (a hook-and-loop strap), such that coolant is passed in close relationship to the skin and flesh of the patient. The paired tubing 16 allows the coolant or ice water to pass to the bladder and, after circulating therethrough, to return to the tank 18. As will become apparent below, the tank 18 is a bulk source of ice water or other appropriate coolant that passes therefrom to the associated bladder and from the bladder back to the bulk source.

With final reference to FIG. 1, it can be seen that a control panel 28 is present on the front of the tank 18 to allow the patient or other operator to program the cold therapy system 10 for the desired treatment to ensure that such treatment is effective for relieving the pain and advancing healing, while not causing ancillary injury or damage to the patient or individual. The control panel 28 allows the selection and control of various parameters to effect proper treatment. While shown as being on the front of the tank 18, it is also contemplated that the controls of the panel 28 may be in the power cord of the system 10 or otherwise as apparent to those skilled in the art.

With reference now to FIG. 2, an appreciation of the structure and operation of the control system 30 of the system 10 may be obtained. As shown, the system 10 may be connected to an AC power source 32 such as available at a wall outlet, This AC voltage is applied to an AC/DC converter 34, the control system functioning off of DC power. A DC battery 36 is interconnected between the control panel 28 and a switch 38, which also is connected to the AC/DC converter 34. With the AC power source 32 also connected to the control panel 28, an operator may select whether the system is to operate off of the AC/DC converter 34 or the battery 36 as then effected by the switch 38. This selection is most preferably mutually exclusive. It is also contemplated that the battery 36 may be a rechargeable battery, recharged by the output of the AC/DC converter 34.

As discussed above, it is desirable that the cold therapy practiced on a patient or other individual be of such nature that the duration can be set, the repetition can be set, and even the effective temperature of the cold therapy may be controlled, To that end, a timer 40 and a duty cycle regulator 42 are provided in series interconnection between the effective power source 32, 36 and the pump 48 to be discussed below.

The order of the series interconnection between the timer 40 and duty cycle regulator 42 is unimportant since the two operate together to perform a logic “AND” function allowing the DC voltage from the appropriate power source to reach the pump 48 only when the timer and duty cycle regulator are both “on.” In other words, the timer and duty cycle regulator act as switches in series interconnection.

The timer 40 may be set for a window of operation by the control panel 28. For example, the timer may be set for one hour. The duty cycle regulator 42 may also be set from the control panel 28 to be “on” and “off” in a series of times, the period of being “on” and the period of being “off” not necessarily being equal, When the switch of the timer 40 and the switch of the duty cycle regulator 42 are both “on,” those switches allow the DC voltage from either the battery 36 or the AC/DC converter 34 to pass to the pump 48 for purposes as will be discussed below.

With continued reference to FIG. 2, it can be seen that a reservoir 44 is maintained within, or defined by, the tank 18, Maintained within the reservoir 44 is an appropriate cooling fluid, most preferably ice water 46. The ice water 46 will typically be of a temperature of approximately 33° F., or slightly higher, until the ice melts. With the tank 18 being insulated, a full melt occurs slowly and ice can be replenished and water removed as desired.

Maintained within the ice water 46 of the reservoir 44 is a pump 48 having a sump tube 50 extending near the bottom of the reservoir 44 such that the pump, when activated, draws water, rather than ice, which could cause plugging.

As shown in FIG. 2, the paired tubing 16 communicates with the reservoir 44 through an appropriate sealed aperture in the wall of the tank 18. This paired tubing 16 includes a cooling tube 16a that is interconnected between the pump 48 and an input side of a bladder of the cooling pad 14. The tubing 16b is interconnected between an output side of that bladder and empties into the reservoir 44, where it mixes with the ice water in that reservoir and thus returns to the temperature of that ice water.

In use, a patient or other operator fills the reservoir 44 with an adequate amount of ice and water 46, Then, on the control panel 28, the patient or other operator programs the timer 40 for the desired period of time for the cold therapy session. The user can then select the desired duty cycle of operation as directed by his/her doctor by appropriately programming the duty cycle regulator 42, anywhere from being continually “on” or being “on” for any selected period of time, such as 50/50, 60/40, 75/25 or the like. The selection of the duty cycle effectively regulates the temperature imparted to the treated area of the user's body. During the “off” period, the temperature of the treated area begins to recover or stabilize since the pump 48 is turned off for lack of power and the cold water no longer circulates.

Thus it can be seen that the various aspects of the invention have been attained by the structure presented above. While in accordance with the patent statutes, only a best known and preferred embodiment of the invention has been presented and described in detail, the invention is not limited thereto or thereby. Accordingly, for an appreciation of the true scope and breadth of the invention, reference should be made to the following claims.

What is claimed is:

1. A programmable cold therapy system for pain relief and to promote healing after an injury or surgery, comprising:
a tank for maintaining a reservoir of cooling fluid;
a pump within said tank and in communication with said reservoir of cooling fluid;
a cooling pad;
a hose assembly interconnecting said cooling pad and pump; and
a control panel interconnecting a power source and said pump, said control panel regulating operation of said pump,

2. The programmable cold therapy system according to claim 1, further comprising a timer interposed between said power source and said pump and setting a first period of time during which said power source may be operatively connected to said pump.

3. The programmable cold therapy system according to claim 2, further comprising a duty cycle regulator interconnected with said timer and setting second periods of time within said first period of time during which said power source is operatively connected with said pump.

4. The programmable cold therapy system according to claim 3, wherein said timer and said duty cycle regulator are connected to said control panel and adapted to be selectively set by an operator to set said first period of time and said second periods of time.

5. The programmable cold therapy system according to claim 4, wherein said power source is an AC power source and said pump is a DC pump, and further comprising an AC/DC converter selectively interconnecting said power source and said DC pump.

6. The programmable cold therapy system according to claim 5, further comprising a DC battery selectively interconnected with said DC pump, said control panel selectively and mutually exclusively interconnecting said battery and AC/DC converter with said pump.

7. The programmable cold therapy system according to claim 4, wherein said cooling pad comprises a compressive bladder having an input and output and hook-and-loop fasteners adapted for securing the cooling pad to particular human body parts.

8. The programmable cold therapy system according to claim 7, wherein said particular human body parts are taken from the group comprising head, back, shoulders, hips, thighs, calves, knees, ankles, wrists and arms.

9. The programmable cold therapy system according to claim 7, wherein said hose assembly comprises a first hose interconnecting said pump with said bladder and a second hose interconnecting said bladder with said reservoir.

10. The programmable cold therapy system according to claim 9, wherein said fluid in said reservoir is cold water generated from a combination of water and ice.

11. The programmable cold therapy system for pain relief and to promote healing, comprising:
a tank having a reservoir of ice water;
a cooling pad having a bladder and selectively attachable to a portion of a user's body;
a pump within said reservoir of ice water;
a first hose interconnecting said pump with said bladder;
a second hose interconnecting said bladder with said reservoir; and
means for operating said pump at a selected duty cycle within a selected window of time.

12. The programmable cold therapy system according to claim 11, wherein said means comprises first and second timers operatively interconnected between a power source and said pump, said first timer establishing said window of time and said second timer establishing said duty cycle.

13. The programmable cold therapy system according to claim 12, wherein said window and duty cycle are programmable from a control panel by an operator.

14. The programmable cold therapy system according to claim 13, wherein said cooling pad is taken from a group configured to cover portions of the human body taken from the group of head, shoulders, arms, wrists, hands, feet, ankles, calves and thighs.

15. The programmable cold therapy system according to claim 14, wherein said cooling pad is held in place on a user by straps and hook-and-loop connectors.
